(19) United States
(12) Patent Application Publication
(10) Pub. No.: US 2004/0177389 A1
(43) Pub. Date: Sep. 9, 2004

(54) Methods

(76) Inventors: David William Silversides, Hyacinthe (CA); Maria Isabelle Daneau, Drummondville (CA); Nicolas Pilon, Valerien (CA); Saffron C Dornan, Cambridge (GB)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/465,913

(22) PCT Filed: Dec. 24, 2001
(86) PCT No.: PCT/GB01/05773

(30) Foreign Application Priority Data
Dec. 28, 2000 (GB) 0031744.6

(51) Int. Cl. A01K 67/00

(52) U.S. Cl. 800/8

(57) ABSTRACT

Methods for controlling sex determination in non-human mammals are provided, including constructs for the production of transgenic mammals and the non-human transgenic mammals themselves.

The present invention relates to novel porcine genetic constructs, and their use in the production of transgenic pigs. The constructs allow for control of the sex of progeny animals.

In mammals, the sex of an individual can be described at three biological levels: genetic or chromosomal sex, gonadal sex, and phenotypic sex. Chromosomal sex is fixed at the time of fertilization. Males are the heterogametic sex, having a genome that contains a single X chromosome and a single Y chromosome, while females are the homogametic sex, having a genome that contains two X chromosomes. Gonadal sex refers to the presence of testes or ovaries in the individual; gonadal sex is established during organogenesis by the process of sex determination, whereby the indifferent gonads are directed to a testicular or ovarian developmental fate. Phenotypic sex refers to whether the animal has the external characteristic male or female body form and results from the developmental process of sex differentiation. In the normal situation there is a tight correlation between these three levels of biological sex to produce functional, fertile and unambiguous male and female animals.

The SRY gene on the Y chromosome is known to initiate testes determination in male mammals, have a dominant positive effect on testes determination, and is equivalent the genetically defined Testes Determining Factor (TDF) (Sinclair, A. H. et al., 1990, Nature, 346:240-4; Gubbay, J. et al., 1990, Nature 346:245-50. Koopman, P. et al., 1991, Nature 351:117-21). The SRY gene is located on an ancient part of Y chromosome common to eutherian, metatherian (marsupials) and protherian mammals Sequences for SRY are poorly conserved between species (Tucker P K and B L, 1993, Nature 364:715-717). The SRY gene is a member of a family of DNA binding proteins characterized by a region known as the high mobility group domain or HMG box. The only homology between the human SRY and mouse SRY genes is within the HMG box.

In mice, expression of SRY is consistent with its role in sex determination. SRY is expressed by pre-Sertoli cells between embryonic day 10.5 (e10.5) and e12.5 (Hacker 95) and is the first gene within the developing gonads which is differentially expressed between the sexes (being absent in females). Studies of gene expression during sex determination have been performed in additional species including sheep, pigs and humans (Payen, E. et al., 1996, Int J Dev Biol 40(3):567-75; Daneau I, et al 1996, Biol Reprod. 55:47-53; Parma, P. et al., 1999, Biol Reprod 61(3):741-8; Hanley N. A. et al., 1999, Mech Dev 91 (1-2):403-407), and although the time course of gene expression is altered, it appears that the relative sequence of events parallels that seen in the mouse.

Little is currently known about the control of SRY gene expression. Sequence comparisons of human and mouse SRY 5′ flanking regions have revealed little homology (Hacker, A. et al., 1995, Development 121:1603-1614), and limited sequence information is available from other species (Daneau I, et al., 1995, Biol Reprod.52(3):591-599; Daneau 1, et al., 1996, Biol Reprod. 55(1):47-53; Margarit, E. et al., 1998, Biochem Biophys Res Commun. 245(2):370-377). There is transgenic evidence that transactivating nuclear proteins required for SRY expression are found equally in XY and XX cells of the undifferentiated gonadal ridge, since the mouse SRY promoter, in the presence of SRY open reading frame, can be functional in both male and female tissues (Koopman, P. et al., 1991, Nature 351′117-21).

Genetic studies in humans have also implicated loci on chromosomes 8, 9 and 17 during the process of sex determination. The gene on chromosome 17 was shown to be SOX-9 (Foster, J. W, et al. 1994, Nature. 372:525-30; Wagner T, et al., 1994, Cell.79(6):1111-20). SOX9 codes for an HMG box protein, related to SRY, that contains a functional transactivating domain. In the human population, haploid deficiency of SOX8, such as by loss of the 3′ transactivating domain of one allele, will result in sex conversion and also anomalies in skeletal development (Sudbeck P, et al., 1996, Nat Genet. 13(2) :230-232). SOX9 shows a sex dependant gonadal expression in the mouse, with expression increasing just after initiation of SRY expression in the developing testes, at the same time that it is decreasing in developing ovaries.

The gene involved in sex determination, sex reversal and gonadal dysgenesis located on chromosome 9 has been identified at the molecular level, and named DMRT1 (Raymond C. S., et al., 1998, Nature. 391:691-695; Veitia, R. et al., 1997, Genomics 41 (2):271-4). This involved homology searches using Drosophila Doublesex (Dsx) and C. elegans Mab-3 genes and human expressed sequence tags (ESTs) (Raymond C. S. et al., 1998, Nature. 391:691-695). DMRT1 is interesting in several respects. It is the first molecule implicated in sex determination that shows sequence conservation between phyla. The avian homologue of DMRT1 is found on the Z (sex) chromosome of chickens and is differentially expressed in the genital ridges of male and female chicken embryos (Raymond, C. S., et al., 1999, Dev Biol. 215(2):208-220; Smith, C. A. et al., 1999, Nature 402:601-602). In addition the reptile homologue of DMRT1 is reported be differentially expressed in embryos developing at male or at female temperatures (Smith, C. A. et al., 1999, Nature 402:601-602). DMRT1 is the only other gene (besides SRY) thus far implicated in mammalian sex determination that appears to have a strictly gonadal pattern of expression (Raymond, C. S., et al., 1999, Dev Biol. 215(2):208-220). The DMRT1 promoter function is currently poorly characterised. DMRT1 expression may occur before that of SRY and thus potentially control the onset of SRY expression (Raymond C. S., et al., 1999, Dev Biol. 215(2)208-220), or after that of SRY, i.e. possibly being controlled by SRY expression (Smith, C.A. et al., 1999, Nature 402:601-602).

Man has modified the phenotypic characteristics of domestic animals through selection of seed stock over many generations ever since animals were domesticated. This has led to improvements in quantitative production parameters such as body size, muscle mass, and milk production. Although it would be very advantageous to direct the sex of offspring, this has proved not to be amenable to selection. Efforts to produce sex selection systems have focussed on the separation of sperm according to their sex chromosome content (semen sexing) or utilizing embryo technologies to transfer embryos of a given sex to recipients (embryo sexing).

The ability to produce single sex litters would be of great benefit to the agricultural industry. For example:

1. Pig producers would be able to take advantage of faster male growth rates in low slaughter weight markets or produce only females in high slaughter weight markets, thus avoiding boar taint problems and the need for castration. Single sex finishing will also make production more efficient. Slaughter plants and processors would benefit from more uniform animals. Finally the breeder would realise efficiencies in selectively producing male or female litters for boar and dam line sales. It has been estimated that the ability to produce single sex litters would be worth more than £10 million per annum in the UK alone

2. Dairy farmers typically replace about 40% of their herd females annually with their female calves. Calves not selected to be retained in the herd (all males and 20% of the females) are sold for meat production (mainly veal). For example semen sexing would guarantee the right number of replacement heifers to be produced while ensuring all other progeny are males sired using beef genetics.

3. In the beef and lamb industries, males are preferred as they have better characteristics than females. Again slaughter plants and processors would benefit from more uniform animals.

Semen sexing also has applications in humans, allowing couples at risk of producing offspring affected by sex-linked genetic disorders, to have daughters by using insemination with selected X sperm. This would be preferable to the current system of amniocentesis and selective abortion to many couples.

Many claims for semen sexing systems have been made over the years based on techniques to physically separate X and Y sperm (see Hossain et al 1998 Arch Androl 40 3-14; Johnson 1996 Dtsch Tierarztl Wochenschr 103 288-291; Windsor et al., 1993 Reprod Fertil Dev 5 155-171 , for reviews). Many patent applications have been filed in this area (eg U.S. Pat. No. 4,362,246, WO84/01265, 1984, WO-A-90/13303, U.S. Pat. No. 5,135,759, WO 90/13315, EP-b-0475936 and WO91/17188). However only fluorescence activated cell sorting (FACS) has so far proved to be an authentic semen sexing system (see Johnson 1996 Dtsch Tierarztl Wochenschr 103 288-291). FACS involves the use of a fluorescent dye that penetrates into the nuclei of sperm cells and binds to the nuclear DNA. When isolated stained sperm are illuminated with UV light, they fluoresce and the amount of fluorescence is proportional to the amount of DNA in that sperm. Because the X chromosome is longer than the Y chromosome, sperm carrying the X chromosome contain more DNA than those carrying the Y chromosome. On this basis FACS is able to discriminate between the two sperm cell types and produce pools of separated sperm of very high purity (>90%).

Although the use of FACS separated sperm is close to commercialization in the cattle industry, it is currently of limited use in the pig breeding industry. This is because the rate of sorting is too slow to be useful in producing sexed AI doses. Currently 3 billion sperm are required per AI dose in the pig and maximum sorting rates are in the order 10 million per hour. This means that FACS sexed semen can only be used in pigs in combination with in vitro fertilization and embryo transfer, neither of which is yet routine in pigs (this also severely limits the application of embryo sexing technologies in the pig). However two groups have produced litters of pigs in this way, both in collaboration with Larry Johnson at the USDA in Beltsville USA, a pioneer of FACS technology (Rath et al 1997 Theriogenology 47, 795-800; Abeydeera and Day 1998 UMC Anim Sci Dept Rep 40-42). Both groups used IVF and surgical embryo transfer. The results of these groups showed that although some 30 embryos transferred, only about 4 survived to term. This work also indicates that embryo sexing in pigs would not be an economically viable option at this time.

Another concern about FACS technology is that the use of DNA binding dyes and a UV laser in the process both potentially damage sperm DNA. Although FACS practitioners claim that animals born using this technique are normal, it is highly likely that the process introduces new mutations. Conception rates following inseminations using FACS separated semen are significantly reduced which supports this view. Thus semen sexing and embryo sexing are not practical possibilities to predetermine the sex of litters in pigs.

Important technical and conceptual developments have occurred in gene regulation and transfer methods with respect to production of transgenic animals. In general, gene promoter sequences taken from a different species have proven useful in properly controlling transgene expression (Overbeek, P. A. et al., 1985, PNAS USA 82:7815-7819; Lira, S. A. et al., 1988, PNAS USA 85:4755-4759). Inducible expression of transgenes has been more difficult, although heavy metal induction of the metallothionein promoter has been exploited (Palmiter, R. D. et al., 1982, Nature 300:611-615). This situation is now changing. The bacterial Lac repressor has been adapted for use in eukaryotic expression systems (Fieck A, et al., 1992, Nuc Acid Res 20(7):1785-1791). Also, the yeast GAL4 DNA binding domain, the herpes simplex VP16 transcriptional activation domain and the progesterone receptor ligand binding domain have been used in combination with the progesterone analog RU486 to develop an inducible activator system for transgene expression in transgenic mice (Wang, Y. et al., 1997, Nature Biotech. 15:239-243). Furthermore, the inducible CYP1A1 promoter has been used in transgenic mice to tightly regulate transgene expression using 3-methylchloranthrene as the inducer (see Campbell et al 1996, Journal of Cell Science 109, 2619-2625). The human herpes simplex VP16 transcriptional activation domain (also called TIF) has been put under the control of the bacterial tetracycline promoter to give successful control of transgene expression in vivo via manipulating tetracycline concentrations (Furth, P. A., et al., 1994, PNAS 91:9302-9306; Baron, U. et al., 1997, Nucl. Acids Res 15(14):2723-2729), or alternatively under the control of insect hormone systems (No, D. et al., 1996, PNAS 93:3346-51). A further development has been the use of bacterial recombinase systems, such as the cre-lox system, in transgenic mice (Lasko, M. et al., 1992, PNAS USA 89: 6232-6236; Orban, R. C., et al., 1992, PNAS USA 89:6861-865). The cre recombinase has also been put under the control of tetracycline responsive promoters to allow temporal control of transgene expression (St-Onge, L. et al., 1996, Res. 24(19), 3875-3877).

Here we propose a novel system making use of transgenic technologies to control the phenotypic sex of non-human mammals. While this system results in non-fertile animals, they do have several advantages listed above. These include the following:

1. the animals of the same phenotypic sex will be uniform, providing advantages to slaughter plants;

2. animals of the same phenotypic sex should reach market weight at about the same time, providing an advantage to the producer;

3. growing phenotypic females will obviate the need to castrate males, providing an animal welfare benefit.

Thus, in a first aspect, the present invention provides a method of providing single-sex offspring in a non-human mammal, which comprises the step of crossing a first transgenic parental animal with a second transgenic parental animal wherein said second transgenic parental animal has incorporated in its genome one or more DNA sequences which alter/adapt the expression of a transgene incorporated in the genome of said first transgenic parental animal, which transgene is involved in determination of sex phenotype.

In the context of the present invention, “alter/adapt” expression encompasses various possibilities, including prevention, enhancement or reduction of expression of the sequence in question. In addition, the alteration or adapting of expression can occur at various levels, including that of transcription, translation and even post-translational modification. Prevention, reduction or enhancement of expression may be achieved by means of one or more recombination events, resulting in removal of the target coding sequence from the animal's genome. The methods of the invention can additionally incorporate the step of providing one or both of the first and second transgenic parental animals.

In one embodiment of this aspect of the invention the method comprises:

(i) providing a first transgenic parental animal which has incorporated in its genome a first DNA sequence involved in determination of sex phenotype and a second DNA sequence which prevents expression of the first DNA sequence; and

(ii) providing a second transgenic parental animal which has incorporated in its genome a DNA sequence comprising gonad specific regulatory sequence(s) and a coding sequence which, when expressed, will inactivate, alter expression of or effect removal of the second DNA sequence present in the first transgenic parental animal.

In a further embodiment of this aspect of the invention the method comprises:

(i) providing a first transgenic parental animal which has incorporated in its genome a DNA sequence involved in determination of sex phenotype flanked by recombination sites; and

(ii) providing a second transgenic parental animal which has incorporated in its genome a DNA sequence comprising gonad specific regulatory sequence(s) and a coding sequence which, when expressed, will effect recombination between the recombination sites flanking the DNA sequence of the first transgenic parental animal.

In particular, the methods of the present invention utilise recombination based systems which operate at the transcriptional level, ie the recombination sites are placed outside the reading frame of the gene.

Thus, the present invention provides the basis for genetic methods for manipulation of animal development and phenotype. More specifically the invention provides the genetic basis for controlled uncoupling of gonadal and phenotypic sex from chromosomal sex resulting in developmental gender conversion.

In one approach, the “lock and key” genetic control mechanism, two lines of animals are engineered. One line, the “locked” line, includes incorporated in the genome a transgene with an open reading frame of choice in a transcriptionally locked or non-functional format. The second line, the “key” line, includes incorporated in the genome a transgene “key” under the transcriptional control of a tissue specific promoter sequence. Mating individuals of the two lines puts the two transgenes in the same genome. The result is that the “key” transgene is expressed in a tissue specific pattern and this then unlocks the “locked” transgene, which becomes transcriptionally active within the same restricted cells and tissues, to cause its desired developmental and phenotypic effects.

Genetic “locking” mechanisms include recombinase systems, such as the cre-lox recombinase system. Within this invention pertaining to modification and control of sex phenotype in domestic animals and in the following examples, directing transgene expression specifically to developing gonads is accomplished by the use of tissue specific promoters including the SRY promoter and the DMRT1 promoter.

As an alternative method, a “molecular scissors” genetic method can be adapted to effect developmental and cell specific genomic excision of genes important for specific developmental processes, such as sex determination. Now one line of animals is genetically altered such that a specific gene has been identified and “marked”, for instance with “lox” sequences. A second line of animals is generated containing a “molecular scissors” transgene consisting of, for example, the cre bacterial recombinase under tight transcriptional control via tissue and developmental specific promoters. Mating individuals of these two lines will cause the “molecular scissors” to be expressed in a tissue and developmentally specific way, causing genomic excission of the marked gene sequences within this cell population. Within the context of the present invention pertaining to the developmental modification of sex phenotype, the targeted genomic DNA sequences could include SRY, SOX9, SF1 or any of the genes involved in sex determination, while the tissue specificity of expression of the molecular scissors molecule could be provided by the SRY promoter or the DMRT1 promoter.

Alternatively, the expression of the site specific recombination system itself could be controlled by the use of a promoter that was activated by an external agent. In this way the ultimate expression of the transgene would be controlled by application of an external agent at a selected time. Examples of such controllable promoters include those from the tetracycline-inducible system (see Forster et al 1999, Nucleic Acids Res 27 708-710), the ecdysone gene (see No et al 1996, Proc Natl Acad Sci USA 93 3346-3351), the RU486-indcuible system (see Wang et al 1997, Nature Biotechnol 15 239-243), the zinc-induced metallothionine gene (see Suppola et al 1999, Biochem J 338 311-316), the CYP1A1 gene (see Campbell et al 1996, J Cell Sci 109 2619-2625) and the Tet inducible system (Huang, et al, Mol. Med. 5(2): 129-37 (1999)). Any promoter that can be induced by an exogenous agent in mammalian cells would serve this purpose.

Genetic constructs for use in engineering transgenic non-human animals for use in the methods of the invention form a second aspect of the present invention. These constructs may comprise one or more DNA sequences involved in the determination of sex phenotype, optionally with one or more DNA sequences which prevent expression of the one or more DNA sequences involved in sex phenotype determination.

Host cells comprising such constructs form a third aspect of the present invention.

Non-human transgenic mammal parental animals, as defined in the first aspect of the invention, themselves form a fourth aspect of the present invention. preferably, such animals are pigs, sheep or cows.

The invention will now be described with reference to the following examples, which should not be construed as in any way limiting the scope of the invention.

The examples refer to the figures in which:

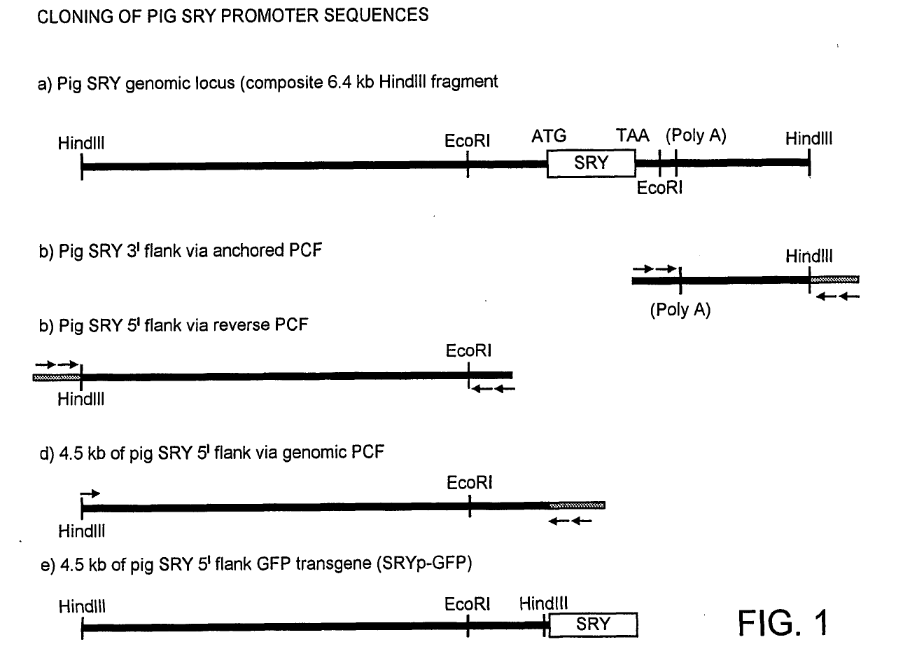

FIG. 1 is a schematic representation of cloning of pig SRY promoter sequences;

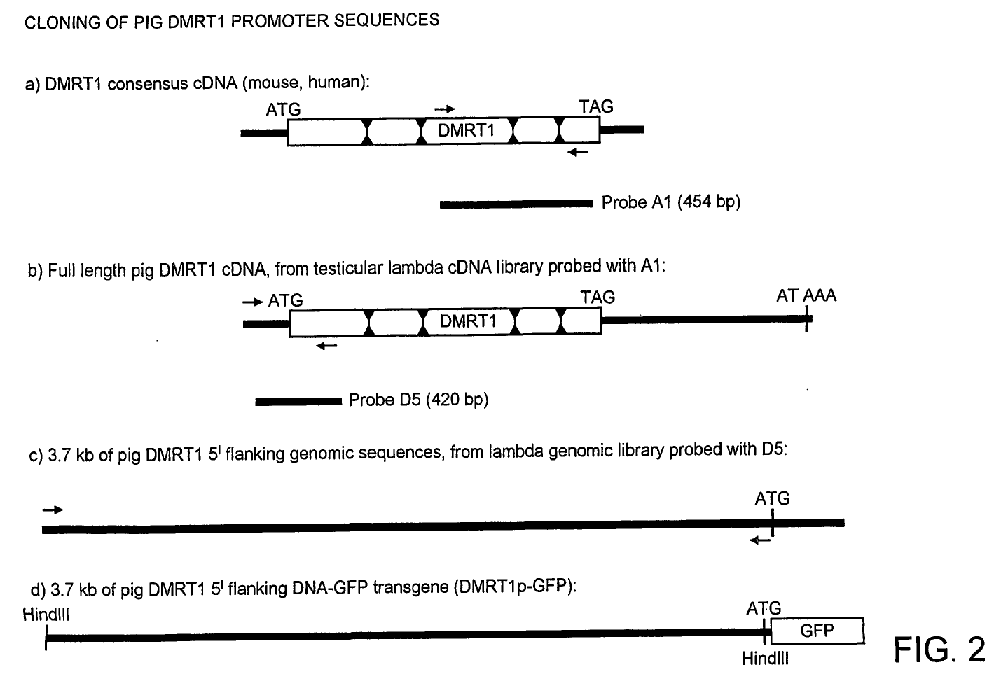

FIG. 2 is a schematic representation of cloning of pig DMRT1 promoter sequences;

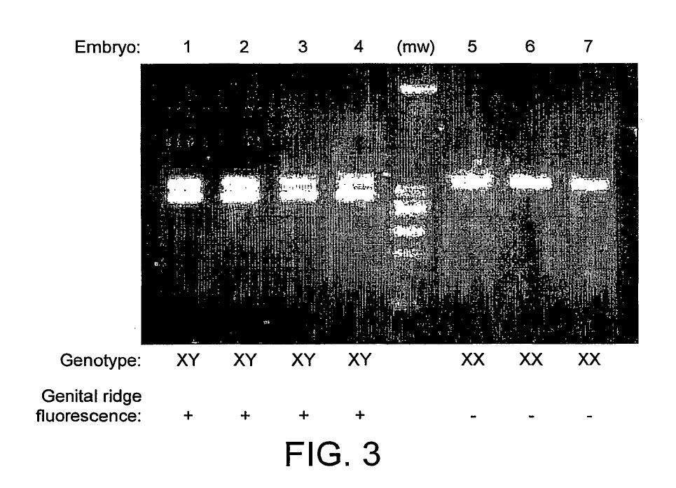

FIG. 3 illustrates PCR sexing of e11.5 mouse embryos transgenic for SRYp-GFP transgene, and correlates the presence of fluorescence in the genital ridges with the XY genotype;

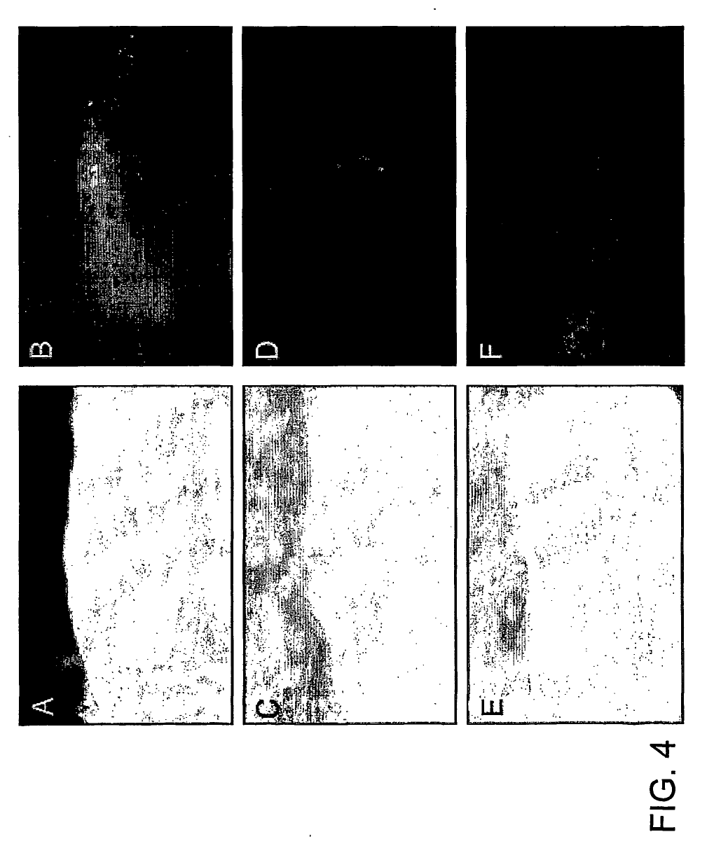

FIG. 4 demonstrates the expression of DMRT1p-GFP transgene expression in porcine genital ridge cells;

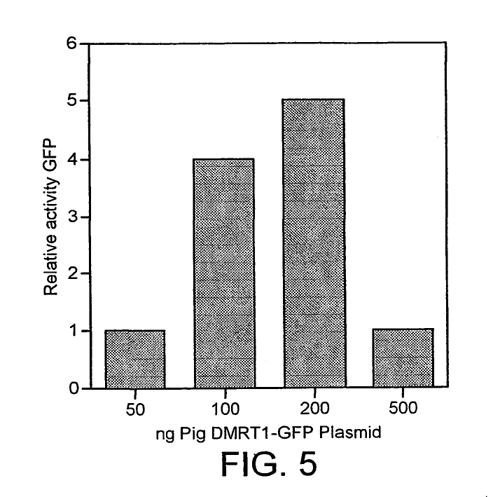

FIG. 5 shows activity of Pig DMRT1 promoter in tissue culture

### EXAMPLE 1

### 5′ Flanking Sequences of SRY Locus and SRYp-GFP Transgene

To initiate the cloning of a 6.4 kb genomic HindIII DNA fragment from the pig SRY locus (Sinclair, A. H. et al., 1990, Nature, 346:240-4), an anchored PCR procedure was used, similar to that previously reported for cloning of a 1.7 kb EcoRI genomic fragment containing the pig SRY open reading frame (Daneau, 1. et al., 1996, Biol Reprod. 55(1):47-53). Briefly, male pig genomic DNA was restricted with HindIII and size fractionated on a 0.8% agarose gel. The bands from 6 to 8 kb were excised and ligated into HindIII cut pBS plasmid (Stratagene), to give a size selected plasmid library. Anchored PCR was then performed, using specific sense primers from the 3′ end of the pig SRY locus (first primer 5′-CACACAAACTGCTTGATTTCG and nested primer 5′-TTCCCGTGATTAGCCATTAAGTACG)(Daneau, 1. et al., 1996, Biol Reprod. 55(1):47-53) and primers derived from plasmid sequences (first primer 5′-AAAGGGGGATGTGCTGCAAGGCG and nested primer 5′-TGGGT AACGCCAGGGTTTTCCCA). A proofreading mix of thermostable polymerases (Expand High Fidelity; Roche) was used for the amplifications. A first PCR amplification used 40 cycles of 45 sec at 95°, 45 sec at 56°, and 4 min at 70°; this was followed by a nested PCR amplification using the same cycling program. This strategy proved successful for amplifying the 3′ end of the genomic HindIII fragment, which was cloned into pGEM®-T vector (Promega), and sequenced.

Anchored PCR was unsuccessful for generating the 5′ end of the pig HindIII fragment, so a reverse PCR method was used. Male pig genomic DNA was again restricted with HindIII, and bands from 6 to 8 kb were excised and then ligated under dilute conditions to favor circularization of fragments. Sense primers were designed from the 3′ end of the genomic HindIII fragment, and antisense primers from pig sequences at the 5′ end of the previously reported EcoRI genomic fragment of the pig SRY locus (Daneau, 1. et al., 1996, Biol Reprod. 55(1): 47-53). A first PCR was performed using the primer pair 5′-AAGCTGATGGTCTCTTGTCTCTGTA and 5′-TTCCTTTCGGCCATTAGAGCACTCA; a second PCR was then performed using the nested primer pair 5′-CTTTCCAGTGCATATATTCCAAAGC and 5′-CGGATGTTATAGAGTTGAATGCTAG. For each amplification, 40 cycles of 45 sec at 95°, 45 sec at 66°, and 4 min at 72° were performed. An amplified band of about 4 kb was ligated into pGEM®-T vector, and sequenced.

Based on sequences obtained for the 5′ end of the pig SRY HindIII fragment, and coding sequences of pig SRY previously reported (Daneau 96), a PCR amplification strategy was performed to obtain 5′ flanking sequences for use in promoter studies. A single sense primer 5′-AAGCTTGGGGAAATCTGTTCAGTA and two antisence primers 5′-GGGGAAATCTGTTCAGTAG and (nested) 5-TTGAAAAGGGGGAGGAAGC were designed. Male pig genomic DNA was used as a template. A first PCR amplification (as described above) was performed followed by a second, hemi-nested PCR amplification under similar conditions. The thermostable polymerase Expand High Fidelity was used. An amplified band of 4.5 kb was then ligated into the plasmid vector pGEM®-T, sequenced and shown to represent the pig SRY 5′ flank due to identity the 3′ end with previously reported sequences (Daneau, I. et al., 1996, Biol Reprod.55(1):47-53).

To construct the reporter transgene for in vitro and in vivo characterization, the 4.5 kb pig SRY 5′ flank was placed in front of a modified enhanced green fluorescent protein reporter sequence (pEGFP-1; Clontech) to give the transgene SRYp-GFP. The pEGF-1 vector had been modified by flanking the transgene with NotI restriction sites to facilitate linearization of the transgene ahead of pronuclear microinjection. The cloning strategy for pig SRY promoter and DNA sequence are presented in FIG. 1 and FIG. 3A, respectively.

Pig DMRT1 Coding and Promoter Sequences and DMRT1 Transgene

To facilitate cloning of pig testicular coding sequences relevant to sex determination, a testicular cDNA expression library was generated in the Lambda Zap cloning vehicle (Stratagene), following the manufacturer's protocols. Heterologous DNA primers were designed based on human and mouse DMRT1 coding sequences (Sense: 5′-ATGGTCATCCAGGATAT TCCTGC); antisense: 5′-TGCTGTCACCAGCAGAGGGCAT, and an RT-PCR performed on adult pig testicular RNA to generate a homologous cDNA probe for pig DMRT1 of 454 bp. The lambda expression library was then screened for DMRT1 coding sequences using the pig cDNA probe, and a full length pig DMRT1 clone was isolated. These sequences were excised in vivo from the lambda vector according to the manufacturer's protocol, to give the plasmid pBK-CMVp-pDMRT1 cDNA. To generate a double stranded DNA probe of 420 bp, based on exon 1 sequence, primers were designed (sense: 5′-GGCTGCAGAGCAGAG GCT; antisense: 5′-TGCACTTCTTGCACTGGCA) and a PCR amplification performed.

This probe was used to screen a pig genomic DNA library (Clontech) for DMRT1 5′ untranslated and promoter sequences. One positive hybridizing clone has provided 2.7 kb of 5′ flanking sequences from the pig DMRT1 gene. Sequences at the 5′ and 3′ ends of the 5′ flanking sequences were used to design primers to amplify this region and the resulting PCR product placed into a GFP reporter plasmid to give the DMRT1p-GFP plasmid. The cloning strategy for pig DMRT1 promoter and DNA sequence are presented in FIG. 2 and FIG. 3B, respectively.

Generation of Transgenic Mice and Matings

Transgenic mice were generated via conventional pronuclear microinjection (Hogan, B. et al., 1994, Manipulating the mouse embryo: A lab manual 2nd ed. Cold Spring Harbor Press, New York), using embryos derived from FVB/N inbred mice (Taketo, M. et al., 1991 PNAS USA 88:2065-2069). A linearized transgene consisting of 4.5 kb of pig SRY 5′ flanking sequences driving GFP was purified from a 1% agarose gel using Sephaglas Bandprep kit (Pharmacia), diluted to a concentration of 1 ng/μl in buffer (Tris 0.5 mM, EDTA 0.1 mM) and then filtered just prior to injection (Ultrafree-MC centrifuge filter, Millipore). The transgene was injected into the pronuclei of single celled mouse embryos. Nine founder transgenic animals were identified, and 7 lines established. Of these 7 transgenic lines, 3 showed strong genital ridge fluorescence when visualized using a Leica MZ FLIII stereomicroscope equipped with epifluorescence and filters sets optimized for GFP (Omega Optical). The remaining 4 lines showed weak or undetectable genital ridge fluorescence. One transgenic line (SRYp-GFP#4) was analyzed in detail for genital ridge expression of GFP, with embryo dissections performed on relevant days of development. Fluoresence was consistently detected in the genital ridges of embryos from day e11.5 to e15.5 (FIG. 4). In the e10.5 genital ridge, fluorescence was inconsistently seen. At e 11.5, about half the transgenic embryos displayed genital ridge fluorescence; these embryos were shown to be XY via PCR based DNA analysis (FIG. 5). From e12.5 to e15.5, the fluorescent gonads were obviously testes via histology, and the cellular pattern of fluorescence reflected the pattern of testicular cords. Thus genital ridge fluorescence was consistently seen with male embryos, but absent in female embryos.

Pig Genital Ridge Cell Lines and Transfection Studies

Pig cell lines were generated from genital ridge cells taken from day e22-23 embryos, when pig SRY is expressed (Daneau, 1. et al., 1996, Biol Reprod. 55(1):47-53). These cells were co-transfected using SV40 large-T antigen and neomycin resistance plasmids. Transfection was performed using Lipofectamine reagent (Gibco). Transformed cells were plated onto 96 well plates and selected for G418 resistance.

One resulting pig genital ridge cell line, named 9E11, was selected for co-transfection studies. Transient transfections were performed in 24 well culture plates using Effectene Reagent (Qiagen). 200,000 cells were plated per well. A reporter plasmid consisting of 2.7 kb 5′ flanking sequences of the pig DMRT1 gene driving the expression of GFP (DMRT1p-GFP) was transfected at 50, 100, and 200 ng per well. Production of fluorescence was quantified by counting 10,000 cells using a fluorescence activated cell sorter. The results of these transfection trials are presented in FIG. 6, where increased fluorescence is correlated with increased plasmid concentration.

### EXAMPLE 2

### XX Genotype Conversion to Male Phenotype

In this case the desired effect is to have offspring all of the male phenotype, i.e. XY animals of a male phenotype and XX animals with a male phenotype. To accomplish this the “lock and key” method is used. In the first instance, the “locked” transgene consists of the SRY open reading frame under the transcriptional control of the SRY promoter but silenced or locked via a LOX-STOP-LOX sequence cassette located just upstream from the normal translational start site. This transgene is used to generate a line of transgenic animals which are bred to homozygocity and are normal in appearance, function and reproduction. The “key” transgene involves the Cre recombinase protein under the transcriptional control of DMRT1 promoter sequences.

This is used to generate a second line of transgenic animals, which are again bred to homozygocity and are normal in appearance, function and reproduction, but which express the Cre recombinase in developing gonads of both sexes. Mating of these two lines results in the Cre recombinase being expressed in the developing gonads of sexes at the time of sex determination. The Cre recombinase will cause excision of the LOX-STOP-LOX cassette, resulting in activation of the SRY transgene in the cells of the genital ridge. In the XY animal, sex determination will not be altered and a male phenotype will result. In the XX animal, the expression of SRY will cause testes determination, which will in turn cause gender conversion resulting in a male phenotype. In such a way, litters of all male phenotype animals will be produced.

In a similar fashion, the “locked” transgene can consist of the SOX9 open reading frame under the transcriptional control of the SRY promoter but silenced or locked via a LOX-STOP-LOX sequence cassette. In a similar fashion, the promoter of the “locked” transgene can consist of a non-tissue specific and strong promoter such as the cytomegalovirus (CMV) promoter. FIG. 7A is presented to illustrate this example.

### EXAMPLE 3

### XY Genotype Conversion to Female Phenotype

In this case the desired effect is to have offspring all of the female phenotype, i.e. XX animals with female phenotype and XY animals with a female phenotype. In the first instance this will be accomplished by using the “lock and key” method. The “locked” transgene consists of antisense sequences of the SRY gene under the transcriptional control of the DMRT1 promoter sequences but silenced (or “locked”) by a LOX-STOP-LOX sequence cassette located just upstream from the antisense sequences. This transgene is used to generate a line of transgenic animals which are bred to homozygocity and are normal in appearance, function and reproduction. The “key” transgene involves the Cre recombinase protein also under transcriptional control of DMRT1 promoter sequences. This is used to generate a second line of transgenic animals which are bred to homozygocity and which are again normal in appearance, function and reproduction, but which express the Cre recombinase in the developing gonads of both sexes at the time of sex determination. Mating of these two lines results in the Cre recombinase being expressed in the developing gonads of both sexes at the time of sex determination. The Cre recombinase will cause excision of the LOX-STOP-LOX cassette, resulting in activation of the antisense SRY transgene in the cells of the genital ridge. In the XX animal, sex determination will not be altered and a female phenotype will result as normal. In the XY animal, the expression of antisense SRY will cause inactivation of the endogenous SRY transcript and a functional inactivation of testes determination, which will in turn cause gender conversion and a female phenotype. In such a way, litters of all female phenotype animals will be produced.

In a similar fashion, the antisense sequences of the “locked” transgene can be antisense SOX9 or antisense SF1. In a similar fashion, the promoter of the “locked” transgene can be a non-tissue specific and strong promoter such as the cytomegalovirus (CMV) promoter. FIG. 7B is presented to illustrate this example.

An alternate approach of XY genotype conversion to female phenotype is via the “molecular scissors” method. In one line of animals, the genomic locus for the SRY gene is “marked” using lox sequences flanking the SRY open reading frame on the Y chromosome. In a, second line of animals, the “molecular scissors” transgene is introduced, consisting of the Cre recombinase protein under transcriptional control of the DMRT1 promoter sequences, and bred to homozygocity. Mating of these two lines results in the Cre recombinase being expressed in the developing gonads of both sexes at the time of sex determination. In the XX animal resulting from this cross, there is no target for the Cre recombinase, and the normal female phenotype results. In the XY animal resulting from this cross, the Cre recombinase will cause excision of the “marked” genomic locus, in this case the SRY gene, interfering with testes determination and resulting in a female phenotype. In such a way, all female phenotype litters of animals will be produced.

In a similar fashion, the “marked” genomic locus can be the SOX9 gene. In a similar fashion, the “molecular scissors” transgene consists of the Cre recombinase under the control of SRY promoter sequences. FIG. 7C is presented to illustrate this example.

1. A method of providing single-sex offspring in a non-human mammal, which comprises the step of crossing a first transgenic parental animal with a second transgenic parental animal wherein said second transgenic parental animal has incorporated in its genome one or more DNA sequences which alter/adapt the expression of a transgene incorporated in the genome of said first transgenic parental animal, which transgene is involved in determination of sex phenotype.

2. A method as claimed in claim 1 which comprises:
(a) providing a first transgenic parental animal which has incorporated in its genome a first DNA sequence involved in determination of sex phenotype and a second DNA sequence which prevents expression of the first DNA sequence; and
(b) providing a second transgenic parental animal which has incorporated in its genome a DNA sequence comprising gonad specific regulatory sequence(s) and a coding sequence which, when expressed, will inactivate, alter expression of or effect removal of the second DNA sequence present in the first transgenic parental animal.

3. A method as claimed in claim 1 which comprises:
(a) providing a first transgenic parental animal which has incorporated in its genome a DNA sequence involved in determination of sex phenotype flanked by recombination sites; and
(b) providing a second transgenic parental animal which has incorporated in its genome a DNA sequence comprising gonad specific regulatory sequence(s) and a coding sequence which, when expressed, will effect recombination between the recombination sites flanking the DNA sequence of the first transgenic parental animal.

4. A method as claimed in claim 2 or claim 3 wherein the gonad specific regulatory sequence is the SRY or DMRT1 promoter.

5. A method as claimed in any one of claims 2 to 4 wherein the DNA sequence involved in sex determination is the SRY, SOX9 or SF1 gene.

6. A method as claimed in any one of claim 2, 4 or 5 wherein the second DNA sequence is a “stop expression” sequence.

7. A method as claimed in claim 6 wherein the “stop expression sequence” is a polyadenylation signal.

8. A method as claimed in any one of claims 2, 4 or 5 wherein the second DNA sequence codes for an antisense RNA molecule.

9. A method as claimed in any one of claims 6 to 8 wherein the “stop expression sequence” or antisense sequence is flanked by sequences for a site specific recombination system.

10. A method as claimed in claim 9 wherein the sequences are loxP or FRT sites, or sequences for site specific recombination systems from bacteriophages lambda or mu, or bacteria such as salmonella.

11. A method as claimed in claim 9 or claim 10 wherein the “stop expression sequence” or antisense sequence can be deleted following expression of a site specific recombinase, which acts upon the flanking sequences flanking the stop expression sites.

12. A method as claimed in claim 11 wherein the site specific recombinase is cre, FLP, or from site specific recombination systems from bacteriophages lambda or mu, or bacteria such as salmonella

13. A method as claimed in claim 11 or claim 12 wherein expression of the site specific recombinase is under the control of regulatory sequences, eg a promoter, which are controllable, such that expression can be activated as desired.

14. A method as claimed in claim 13 wherein expression is induced following the application of a specific inducer to the animal, for example in its feed or by intravenous injection.

15. A method as claimed in claim 13 or claim 14 wherein the regulatory sequences are provided by a promoter which is the promoter from the CYP1 A1 or CYP 2B1 gene or by one or more tet-responsive elements (TRE), and induction is achieved using PAH, TCDD, beta NF, PCBs, 3-mc or doxycycline.

16. A method as claimed in any one of claims 1 to 15 wherein one or both of the transgenes is/are integrated into either the X or Y chromosome by means of sequences from expressed, but non-essential, regions of the X or Y chromosome respectively in the target species.

17. A method as claimed in any one of claims 1 to 16 wherein the transgenes are integrated into the genome of embryonic stem cells.

18. A method as claimed in claim 17 wherein selected transgenic embryonic stem cell lines are injected into morulae or blastocysts and the manipulated embryos transferred to suitable recipients.

19. A method as claimed in claim 18 which results in chimaeric animals which are capable of transmitting the transgene in their germ line.

20. A method as claimed in any one of claims 1 to 16 wherein the transgenes are integrated into the genome of totipotent cells in tissue culture.

21. A method as claimed in claim 20 wherein selected transgenic totipotent cells are used as nuclear donors in a nuclear transfer procedure.

22. A method as claimed in claim 21 wherein transgenic reconstructed embryos are transferred to suitable recipients.

23. A method as claimed in any one of claims 1 to 16 wherein the transgene is integrated into one of the sex chromosomes of fertilised eggs following pronuclear injection, lipofection, electroporation or transfection.

24. A method as claimed in claim 23 wherein manipulated embryos are transferred to suitable recipients.

25. A method as claimed in claim 24 which results in chimaeric animals which are capable of transmitting the transgenes in their germ line.

26. A method as claimed in any one of claims 1 to 25 wherein the non-human mammal is a pig, cow, sheep, goat, rabbit or mouse.

27. A non-human mammal produced according to a method as claimed in any one of claims 1 to 25.

28. Progeny of a non-human mammal as claimed in claim 27.

29. A transgene construct as defined by any one or more of the feature of claims 1 to 16.


SEQUENCE LISTING (15 sequences)

SEQ ID NO: 1 - DNA, 21 bp, Artificial Sequence
  Primer
cacacaaactgcttgatttcg

SEQ ID NO: 2 - DNA, 25 bp, Artificial Sequence
  Primer
ttcccgtgattagccattaagtacg

SEQ ID NO: 3 - DNA, 23 bp, Artificial Sequence
  Primer
aaagggggatgtgctgcaaggcg

SEQ ID NO: 4 - DNA, 23 bp, Artificial Sequence
  Primer
tgggtaacgccagggttttccca

SEQ ID NO: 5 - DNA, 25 bp, Artificial Sequence
  Primer
aagctgatggtctcttgtctctgta

SEQ ID NO: 6 - DNA, 25 bp, Artificial Sequence
  Primer
ttcctttcggccattagagcactca

SEQ ID NO: 7 - DNA, 25 bp, Artificial Sequence
  Primer
ctttccagtgcatatattccaaagc

SEQ ID NO: 8 - DNA, 25 bp, Artificial Sequence
  Primer
cggatgttatagagttgaatgctag

SEQ ID NO: 9 - DNA, 24 bp, Artificial Sequence
  Primer
aagcttggggaaatctgttcagta

SEQ ID NO: 10 - DNA, 19 bp, Artificial Sequence
  Primer
ggggaaatctgttcagtag

SEQ ID NO: 11 - DNA, 19 bp, Artificial Sequence
  Primer
ttgaaaagggggaggaagc

SEQ ID NO: 12 - DNA, 23 bp, Artificial Sequence
  Primer
atggtcatccaggatattcctgc

SEQ ID NO: 13 - DNA, 22 bp, Artificial Sequence
  Primer
tgctgtcaccagcagagggcat

SEQ ID NO: 14 - DNA, 18 bp, Artificial Sequence
  Primer
ggctgcagagcagaggct

SEQ ID NO: 15 - DNA, 19 bp, Artificial Sequence
  Primer
tgcacttcttgcactggca